(19) United States
(12) Patent Application Publication
(10) Pub. No.: US 2008/0103117 A1
(43) Pub. Date: May 1, 2008

(54) Treatment Product for Animals and Means for Preparing Same

(75) Inventor: Christian Cardon, Brugge (BE)

Correspondence Address: DENNISON, SCHULTZ & MACDONALD, 1727 KING STREET, SUITE 105, ALEXANDRIA, VA, 22314, US

(73) Assignee: CARDON PHAMACEUTICALS NV, Oostkamp (BE)

(21) Appl. No.: 11/722,773

(22) PCT Filed: Dec. 28, 2005
(86) PCT No.: PCT/IB05/03896

§ 371 Date: Jul. 5, 2007

(30) Foreign Application Priority Data
Dec. 31, 2004 (FR) 0414116

(51) Int. Cl. A61K 31/60; A61K 31/155; A61K 31/216; A61K 31/4164

(52) U.S. Cl. 514/165; 514/385; 514/531; 514/635

(57) ABSTRACT

Product for the treatment of household pets as having need of a therapeutic treatment, characterised in that it comprises, in separate form and intended to be mixed:

a non-foaming base suited to constituting a galenic form for topical application and/or dermatological treatment, and

a concentrated preparation of at least one component with reduced biological activity and suited to acquiring a maximum biological activity in relation to the desired treatment, under the effect of an appropriate change to the pH, advantageously to pH 6-7,

whereas said at least one activable component with reduced biological activity is itself premixed with at least one foaming agent and as an option at least one emulsifier in said concentrated preparation, and

whereas the pH of said concentrated preparation is either less than around 5 or more than 8, with as an option adjustment to this value by means of an appropriate buffer.

### FIELD OF THE INVENTION

The present invention concerns the field of treatments for animals, in particular for household pets. More specifically, it concerns means for procuring treatments for household pets adapted to the needs of each one at a given time, preferably as a function of a diagnosis performed beforehand according to conventional methods.

### STATE OF THE PRIOR ART

Shampoo formulations containing substances with therapeutic effect for the dermatological treatment of household pets are known. Such formulations are standardised and cannot suitably treat individual animals for disorders specific to each of them.

Moreover, U.S. Pat. Nos. 5,842,441 and 5,918,568 (the second stemming from the same application as the first, by division) describe a system for the treatment of household pets, comprising in a first recipient an aqueous base with detergents for shampoo and in at least one second recipient one or several medicinal substance concentrates. Said medicinal substance concentrates are not combined with detergent agents and are intended to be transferred via a manual pump assembled on said second recipient into said first recipient, which must then be opened or uncapped to pour the concentrate(s) into said first recipient. The system is based on the supply of individualised concentrates, each comprising a single substance with medicinal activity. The dose of each of these concentrated substances with medicinal activity introduced into the base for shampoo is then predetermined and is a function of the pumping characteristics of said manual pump, which sets the unit values and their multiples.

### DESCRIPTION OF THE INVENTION

It has now been found that one can produce in a significantly more reliable and modular manner products for treating household pets having preventive and/or curative properties vis-à-vis the illnesses or disorders that can affect these animals, by combining, in suitable quantities according to the requirements:
a non-foaming base for topical application and/or dermatological treatment with
a non-active component suited to becoming biologically, therapeutically and/or dermatologically active under the effect of an appropriate pH change, in premix with at least one foaming agent and as an option at least one emulsifier, whereas the pH of said concentrated preparation is less than around 5-6 or is adjusted to be less than around 5-6 by means of an appropriate buffer.

### DETAILED DESCRIPTION OF THE INVENTION

The first subject of the invention is therefore a product for treating household pets diagnosed as having need of a therapeutic treatment, and comprising in respective suitable quantities according to the requirements, in separate form and intended to be mixed:
a non-foaming base suited to constituting a galenic form for topical application and/or dermatological treatment, and
a concentrated preparation of at least one component with reduced biological activity and suited to acquiring a maximum biological activity (in other words becoming biologically, therapeutically and/or dermatologically active) in relation to the desired treatment, under the effect of an appropriate modification of the pH to a value of around 6-7, whereas said at least one activable component with reduced biological activity is itself premixed with at least one foaming agent and as an option at least one emulsifier in said concentrated preparation, and whereas the pH of said concentrated preparation is either less than around 5 or more than 8, with as an option adjustment to this value by means of an appropriate buffer.

On contact with the skin, which has a pH of around 6, said product has preventive and/or curative properties vis-à-vis the illnesses or disorders that can affect animals.

A further subject of the invention is means for the preparation of medicated products for animals, comprising at least one host recipient partially filled with a non-foaming base constituting an appropriate galenic form, and at least one source recipient containing in concentrated form at least one precursor of agent with therapeutic and/or dermatologic activity, as well as an independent dosing device for the transfer of a selectionable quantity of said precursor of agent with therapeutic and/or dermatologic activity and foaming agent, from a source recipient to a host recipient.

“Partially filled” is herein taken to mean that the recipient comprises, above the product that it already contains, a sufficient available space for the introduction of a desired quantity of at least one concentrate according to the present invention.

The product according to the invention thus comprises a non-foaming base, such as for example a support base for shampoo or any other type of support for dermatological application, in the form of aqueous solution, emulsion, lotion, cream, etc.

This base composition for dermatologic preparation is supplied in a recipient of appropriate size to leave free a volume at least sufficient to receive a desired quantity of concentrate to be added to it. Said recipient advantageously comprises marks indicating volumes and/or dosages, as well as systems of labels, preferably self-adhesive, making it possible to visualise at any time the contents of the recipient after each of the operations described later.

“Concentrate” is herein taken to mean any composition of an appropriate solvent or diluent with a majority proportion of a physiologically acceptable substance and suitable for a therapeutic aim. In practice, the proportion by weight of the activable substance(s) with reduced biological activity in such a concentrate is more than 50%, advantageously more than 60% and more preferably around 75% or more, compared to the concentrate as a whole.

The concentrate(s) presented separately from the said base for dermatological preparation and intended to be mixed with said base in proportions and according to predetermined choices on a case by case basis are, as indicated above, each intended for a specific treatment, and may be combined with each other, if necessary.

“Active” herein designates a compound or a mixture of compounds efficacious in relation to a chosen biologic, therapeutic, and/or dermatologic aim.

The substantially equivalent expressions “with reduced biological activity and suited to acquiring a maximum biological activity”, “with reduced biological activity and suited to becoming biologically, therapeutically and/or dermatologically active” or “with activable reduced biological activity”, herein designate a compound or a mixture of compounds that is not substantially active from a biologic, therapeutic and/or dermatologic point of view under the pH conditions concerned, and which acquires all of its biological activity under the conditions indicated.

The therapeutic and/or dermatologic aims concerned by these concentrates may be, for example, the treatment of a pilous system, a bacterial infection, infestations by fungi, a seborrhoeic condition, or other conditions known to those skilled in the art.

The concentrates that may be introduced into and mixed with a base according to the invention comprise, apart from at least one foaming agent, if necessary an emulsifier, as well as, as an option, at least one buffer, chosen among conventional buffers, but also as an option all other conventional additives, such as for example fragrances, thickeners, colourants, agents conferring a pearly appearance, preservatives, and others.

Under one aspect, the invention further concerns means for the preparation of such a personalised treatment product mainly intended for the treatment on a case by case basis of dermatological illnesses of household pets, said means comprising:
at least one host recipient comprising a non-foaming base constituting an appropriate galenic form, as well as a sufficient available volume for the introduction of a desired quantity of at least one other component, in practice an empty volume at least equal to the volume occupied by said non-foaming base, and
at least one source recipient containing at least one precursor of agent with therapeutic activity in concentrated form, as well as
an independent device, preferably a syringe, for drawing off and transferring a predetermined and dosed quantity of said concentrate of precursor of agent with therapeutic activity and foaming agent, from a source recipient to at least one host recipient.

As regards the recipient known as source recipient, which is that from which is withdrawn a dosed quantity of the concentrate to be mixed with an appropriate base contained in at least one host recipient, it is provided according to the invention to advantageously equip it with a system of labels suitably attached and divided over all or part of their surface into detachable zones, advantageously self-adhesive, in order to make the labelling corresponding to a quantity that has just been withdrawn gradually disappear, to only leave showing the quantity or quantities that remain available in the source recipient for any subsequent withdrawals. In this respect, it is recommended to place the labels or fractions of labels on the wall of the recipients at levels such that the upper edge of the label or the remaining fractions of label is substantially at the level of the liquid remaining in the recipient.

One may thus preferably, by way of non limiting example, have a label that can be divided along horizontal cutting lines, and in which the successive fractions of the label bear from top to bottom indications of volumes on a sliding scale, such as for example: 10 ml of (precursor of agent with therapeutic activity), 9 ml of (id.), 8 ml of (id.), 7 ml of (id.), 6 ml of (id.), etc., if unit withdrawals of 1 ml are provided for.

Said same detachable label may, once removed from the source recipient, be affixed to a host recipient, if so desired.

The host recipient is advantageously partially filled up to a predetermined level or with a predetermined volume of non-foaming base. After introduction of one or several appropriate quantities of chosen concentrate(s), one fixes preferably on at least one of its faces visible to the user a label comparable to that described above, also advantageously divisible into fractions indicating the volume remaining in the recipient after each use for the treatment of an animal according to the invention.

In the embodiment that comprises a syringe as means for the transfer in dosed quantity of extracts of concentrate from the source recipient(s) to the host recipient, it is advantageous that said respectively host and source recipients comprise a perforable and self-closing sealing system. One may thus without tedious handling withdraw a desired quantity of concentrated product and transfer it into a host recipient without having to open the recipients, which favours the preservation of the integrity of the products or compositions concerned.

Such a syringe is a preferred means, given that it procures a reliable and precise means for the withdrawals of concentrated product, even when it is the case when this product comprises a foaming agent, and then further procures a reliable and practical means for the introduction of the same dosed quantity, or even if required a determinable fraction of it, into one or several host recipients where the product introduced is mixed with the base already contained in said recipient, if necessary with agitation, mixing or any appropriate mixing technique.

It is preferable to employ single use syringes.

The product resulting from such a mixture is a homogeneous product, at least for the time during which this product is made available for final use. Indeed, due to the very concept on which the invention is based, it is not necessary, or even judicious, to prepare large quantities of the product intended for a treatment, and consequently it is not expected to have to conserve said product over a long time period. On the other hand, it is preferable and strongly recommended to have prepared quantities of such a treatment product for use over a short or medium time, and to return to the veterinary surgeon's or pharmacist's premises to obtain the product afresh, but better suited for pursuing the treatment or even the treatment of after effects of said treatment or other symptoms that have appeared in the meantime.

In practice, it is when the value of the pH of the treatment product according to the invention is taken to a value greater than around 5 or less than around 8, and preferably during the application of said product on the skin (which has a pH of around 6), that the components with reduced biological activity become biologically, therapeutically and/or dermatologically active in the sense of the present invention.

According to the invention, different compositions may be added to a same support base, which is particularly useful if the dermatologic disorder may be attributed to different causes. Also, from a same withdrawal of active and/or activable concentrate, one may carry out, if so desired, an addition of a defined fraction of said concentrate into a first support contained in a first host recipient (for example for shampoo) and the rest into a second support, contained in another host recipient (for example to obtain a cream or a lotion).

Thus, according to the invention, the veterinary surgeon may adapt a treatment as a function of the dermatologic disorder that he has been able to observe during his diagnosis, and thereby personalise the treatment but also combine different medications, while at the same time have the choice of the best support (for example a shampoo rather than a cream, or vice versa) for the single or combined treatment chosen.

By way of purely illustrative examples, one has therefore, according to the invention, prepared a cream formulation with chlorhexidine for topical application by incorporating in 100 g of base for cream, with appropriate mechanical mixing, 1 ml of a concentrate of chlorhexidine precursor, namely a 1% concentrate of chlorhexidine in the form of chlorhexidine digluconate.

One has also obtained a formulation for shampoo with 1% of chlorhexidine by adding 1 ml of concentrate of active material precursor, further comprising an appropriate traditional foaming agent, in a host recipient containing 250 ml of water.

Examples of activable substances that may be present in the concentrates or in which the precursors may be placed in the form of concentrates according to the invention are particularly the following or derivatives thereof and/or physiologically acceptable salts: chlorhexidine, permethrin, climbazole, benzoyl peroxide, salicylic acid, ethyl lactate (decomposed into ethyl alcohol and lactic acid on contact with the skin), chlorhexidine-phenoxyethanol combination, as well as allergens.

The foaming agent may, for its part, be chosen among conventional compounds, known to those skilled in the art for such use, or appropriate mixtures of such agents.

In practice, it turns out to be advantageous that the proportion of aforesaid concentrate introduced into the base according to the invention is around 1 ml for 50 ml, 250 ml or even 500 ml of base or support. This corresponds to a proportion of around 1% to 2.5% by weight of concentrate compared to the total weight of the product ready for the recommended treatment.

By way of illustrative examples, the following active compounds and the following proportions are recommended:

For a bacterial infection, lotions with 1 ml of chlorhexidine-phenoxyethanol concentrate in 100 ml of topical lotion.

For a parasitic infection, shampoo with 1 ml of permethrin concentrate in 250 ml of water.

For a fungal infection, cream with 1 ml of climbazole concentrate in 100 g of cream.

For a dry seborrhoea, one recommends a combination of climbazole, ethyl lactate and salicylic acid, whereas for a greasy seborrhoea one recommends using an appropriate combination of benzoyl peroxide, climbazole and salicylic acid.

The detergents (soaps or non-soaps, the latter being preferred) used in the composition of the concentrate according to the invention represent advantageously around 20% to 75% of the concentrate. The detergents thus used may be chosen as appropriate by those skilled in the art on the basis of their knowledge in this respect. They are in particular anionic detergents, such as sodium or potassium salts of higher fatty acids (for example sodium lauryl sulphate); cationic detergents (for example quaternary ammonium salts); amphoteric detergents (for example beta-amino propionates and alkyl dipropionates).

Apart from the principal products indicated above, the products according to the invention may comprise, as an option, each time in conventional quantities for this type of application, colourants, fragrances, thickeners, solvents, opacifiers, foaming adjuvants, conditioning agents, preservatives, buffer substances, antistatic agents, and other conventional additives.

In a general manner, the process for the selection of a product according to the invention is as follows:

A veterinary surgeon is faced with a request to treat a specific household pet that he has been told has coat and/or skin problems. He carries out the diagnosis of the animal and determines the cause of the problems (seborrhoea, pyodermia, eczema, hyperkeratosis, ichtyosis, pruritus, squamation, psoriasis) and the need for a treatment.

These dermatological problems are linked to a secondary infection reported by the animal's owner. However, in most cases an underlying primary infection is the real cause of the problems in question. Traditionally, when using a technique of the prior art, the veterinary surgeon then prescribes at least two dermatological treatments based on substances with different therapeutic effects, which puts off the animal's owner and may lead to the non-application of the treatment or an application of only one of them, which is unsuitable. Thanks to the recommended means according to the invention, a mixed treatment may be prescribed and, since it does not constitute a difficulty or a source of concern as a multiple treatment would provoke, it is easily accepted and more efficacious since properly applied.

Other uses, as well as other embodiments of the product and/or means for its application are also possible, without going beyond the context of the invention and its scope, defined by the appended claims, which must be interpreted in the light of the preceding description.

1. Product for the treatment of household pets diagnosed as having need of a therapeutic treatment, characterised in that it comprises, in separate form and intended to be mixed:
a non-foaming base suited to constituting a galenic form for topical application and/or dermatological treatment, and
a concentrated preparation of at least one component with reduced biological activity and suited to acquiring a maximum biological activity in relation to the desired treatment, under the effect of an appropriate change of the pH, advantageously to pH 6-7, whereas said at least one activable component with reduced biological activity is itself premixed with at least one foaming agent and as an option at least one emulsifier in said concentrated preparation, and
wherein the pH of said concentrated preparation is less than around 5 or more than 8, with as an option adjustment by means of an appropriate buffer.

2. Product according to claim 1, characterised in that the non-foaming base is in the form of aqueous solution, emulsion, lotion or cream.

3. Product according to claim 1, characterised in that the proportion of concentrate is around 1% to 2.5% by weight compared to the total weight of the product ready for the recommended treatment.

4. Product according to claim 1, characterised in that the concentrates intended to be introduced into and mixed with said base comprise, apart from at least one foaming agent, an emulsifier and, as an option, other conventional additives, such as fragrances, thickeners, colourants, agents conferring a pearly appearance, and preservatives.

5. Means for the preparation of medicated products for household pets, characterised in that they comprise at least one host recipient partially filled with a non-foaming base constituting an appropriate galenic form, and at least one source recipient containing in concentrated form at least one precursor of agent with therapeutic and/or dermatologic activity, as well as an independent dosing device for the transfer of a selectionable quantity of said concentrate of precursor of agent with therapeutic activity and foaming agent, from a source recipient to a host recipient, whereas said precursor of agent with therapeutic activity is maintained at a pH<5 or pH>8 up to its activation at pH 6-7 for use.

6. Means according to claim 5, characterised in that the source recipient comprises marks indicating volumes and/or dosages, as well as systems of labels, preferably self-adhesive, making it possible to visualise, at any time, the contents of said recipient.

7. Means according to claim 5, characterised in that they comprise at least one source recipient equipped with a system of labels suitably attached and divided over all or part of their surface into detachable zones, advantageously self-adhesive, in order to make the labelling corresponding to a quantity that has just been withdrawn gradually disappear, to only leave showing the quantity or quantities that remain available in the source recipient for any subsequent withdrawals.

8. Means according to claim 7, characterised in that the labels or fractions of labels on the wall of the recipients are at levels such that the upper edge of the label or the fractions of label remain substantially at the level of the liquid remaining in the recipient.

9. Means according to claim 5, characterised in that said label can be divided along horizontal cutting lines, and in which the successive fractions of label bear from top to bottom indications of volumes on a sliding scale.

10. Means according to claim 8, characterised in that said detachable label is, once removed from the source recipient, affixed to a host recipient.

11. Means according to claim 8, characterised in that one also attaches onto at least one of the faces visible to the user such a label, advantageously divisible into fractions indicating the volume remaining in the recipient after each use.

12. Means according to claim 5, characterised in that said respectively host and source recipients comprise a perforable and self-closing sealing system.

13. Means according to claim 5, characterised in that the activable substances present in the concentrates or in which the precursors may be placed in the form of concentrates are chosen among the following or derivatives thereof and/or physiologically acceptable salts: chlorhexidine, permethrin, climbazole, benzoyl peroxide, salicylic acid, ethyl lactate, chlorhexidine phenoxyethanol combination, as well as allergens.

14. (canceled)
